(19) United States
(12) Patent Application Publication
(10) Pub. No.: US 2024/0290490 A1
(43) Pub. Date: Aug. 29, 2024

(54) WISDOM BASED DECISION SYSTEM

(71) Applicant: Cercle.ai, Inc., Frisco, TX (US)

(72) Inventor: Juan Carlos RIVEIRO INSUA, Menlo Park, CA (US)

(21) Appl. No.: 18/581,111

(22) Filed: Feb. 19, 2024

Related U.S. Application Data
(63) Continuation-in-part of application No. PCT/US2023/030440, filed on Aug. 17, 2023.
(63) Continuation-in-part of application No. 18/235,050, filed on Aug. 17, 2023, now abandoned.
(60) Provisional application No. 63/398,815, filed on Aug. 17, 2022.
(60) Provisional application No. 63/398,815, filed on Aug. 17, 2022.

(51) Int. Cl. G16H 50/20; G06T 7/00; G16H 30/40; G16H 50/30

(52) U.S. Cl. CPC G16H 50/20; G06T 7/0016; G16H 30/40; G16H 50/30; G06T 2207/10056; G06T 2207/30044

(57) ABSTRACT

A decision system includes a dynamic biomedical graph configured to provide multiple functions. Through dynamic variation of the biomedical graph structure a more optimal segregation of a diverse set of inputs can be identified. A decision process by which a conclusion is reached can be viewed as a path through the biomedical graph. In contrast with a typical “black box” neural network, this approach clarifies how and why a particular conclusion was reached. Such clarification can lead to identification of new and/or unexpected relationships between input data and resulting conclusions, and also provide confidence that the conclusion was based on a sensible decision process. The decision system is applied, as an example, to medical decisions made in In Vitro Fertilization (IVF).

### CROSS-REFERENCE TO RELATED APPLICATIONS

This application claims benefit and priority of PCT/US23/30440 filed Aug. 17, 2023; PCT/US22/40782 filed Aug. 18, 2022; Ser. No. 18/235,030 filed Aug. 17, 2023; U.S. Pat. No. 63,234,555 filed Aug. 18, 2021 and Ser. No. 63/398,815 filed Aug. 17, 2022. The disclosures of the above patent applications are hereby incorporated herein by reference.

### BACKGROUND

### Field of the Invention

The invention is in the field of artificial intelligence, some applications include the use of artificial intelligence in medical diagnostics and in decision making.

### Related Art

Artificial intelligence has been used in the field of image processing including the analysis of medical images.

### SUMMARY

A biomedical graph is combined with one or more neural network to produce an adaptable AI system. The biomedical graph provides a decision path in which a substantial number of inputs can be analyzed in a series discrete steps. The structure, properties, parameters and connections within the biomedical graph result in a segregation of the inputs into manageable groups of inputs. The neural networks may then be used to process these manageable groups of the inputs at each of the discrete steps, each discrete step being represented by a node or set of nodes of the biomedical graph. Segregation of inputs permits a substantial number of inputs of different types. In addition, the structure of the biomedical graph makes the system adaptable and allows for the discovery of new relationships and associations within the inputs.

At a structural level, the biomedical graph satisfies at least two functions. First, though dynamic variation of the biomedical graph structure (nodes, properties, parameters and edges) and building knowledge by organizing the data in the graph structure, optimal segregation of inputs and decision processes may be found. This function is performed using structure evolution logic as described elsewhere herein. Second, the decision process by which a conclusion is reached can be viewed as a path through the biomedical graph. In contrast with a typical “black box” neural network, this path clarifies how and why a particular conclusion was reached. Such clarification can lead to identification of new and/or unexpected relationships between input data and resulting conclusions, and also provide confidence that the conclusion was based on a sensible decision process.

These conclusions can be reached even if the biomedical graph is incomplete. For example, if the biomedical graph has missed edges, links, parameters, properties or even missing nodes. Further, the biomedical graph is resilient with regard to incorrect input data. If the input data includes errors, some part of the biomedical graph can drive toward wrong conclusions. However, the system is optionally configured to self-learn to detect the incorrect data and thus still reach correct conclusions. These abilities are optionally managed by assigning a confidence level to some or every parameter, property, node and edge in the knowledge. The confidence levels may be important to not only the decision process but also in the self-learning processes. High confidence means that the data is validated by doctors and technicians but also the score of confidence is the result of a function that manage the reward function of a reinforcement learning system that increases the confidence score the more we use that data with a successful output. What is the successful output? it is the 2^{nd} score that is associated to every element of the knowledge based, basically measures the involvement of that parameter, property, edge or node with a successful outcome of the knowledge=successful conclusion; in the case of IVF, it would mean that the successful outcome is a birth of a healthy baby, but there could other successful conclusions/predictions. The higher score is basically for those that are always associated only for successful conclusions. As we have to manage significant about knowledge/data, together with their 2 score levels the complexity to reach conclusions grows exponential, so that is the re reason to segment the graph into multiple graphs or groups of data so the system can run analysis in parallel and independent of each other but requiring a multi-layer approach that will consolidate conclusions, self-learning, reinforcement learning to keep a single and coherent knowledge based/biomedical graph. Various embodiments include multiple neural networks, optionally trained using reinforcement learning techniques. These neural networks may be configured to analyze single or multiple factors (e.g., biomedical characteristics and/or data elements) to identify improved approaches to IVF and/or other medical conditions discussed herein.

In some embodiments, the systems and methods disclosed herein are used to minimize the number of embryos that are discarded. For example, an embryo may be created from an egg and sperm and then evaluated for success probability in IVF. Only if the probability is below a threshold is another embryo created. In some embodiments, the probability of success for two or more embryos are evaluated and a number of embryos implanted together (e.g., in the same IVF cycle) is based on a targeted multiplicity of the pregnancy. For example, the number and/or identity of embryos implanted in the mother may be selected such that there is a 15% chance of no pregnancy, 75% chance of a single fetus, an 8% chance of twins and a 2% chance of triplets. Given an estimated probability for each embryo, a caregiver can select embryos for implantation, and/or the number thereof, to target a desired probability distribution of outcomes. The systems disclosed herein optionally include prediction logic configured to perform these calculations and guide embryo selection.

At an individual node level, optionally discrete neural networks provide outputs based on inputs. However, because of the structure evolution logic, the inputs considered by a particular neural network at a particular node of the biomedical graph are a segregated subset of available inputs. During development of the biomedical graph the inputs at individual nodes may be varied and particular neural networks may be retrained (without retraining the entire system).

Wisdom is achieved when the structure of the biomedical graph is evolved to identify new decision paths and input segregation schemes. For example, if the biomedical graph is evolved to include new decision processes to facilitate a medical diagnosis, then new medical “wisdom” is produced.

The dynamic biomedical graph discussed herein may be applied to a wide variety of applications. However, for the purposes of example, the systems and methods described are focused on medical diagnosis and decision making. In particular, the application of a dynamic biomedical graph to IVF (In Vitro Fertilization) decision making is demonstrated. Initial results demonstrate a substantial improvement in the success of IVF. This improvement is based, at least in part, on an ability to consider a wide variety of inputs and segregate these inputs in an at least partially optimized combination.

Various embodiments include a medical decision system comprising: an input module configured to receive diagnostic data including image data and clinical data; non-transient storage configured to store: a biomedical graph configured for reaching a medical decision based on the diagnostic data, edges of the biomedical graph being configured to represent a decision path through the biomedical graph, nodes of the biomedical graph being configured to receive a segregated subset of the diagnostic data and to provide outputs from a respective node based on analysis of the received segregated subset, one or more neural networks associated with nodes of the biomedical graph, and the diagnostic data; decision logic configured to navigate the biomedical graph based on the diagnostic data and the neural networks, navigation of the biomedical graph resulting in a viability score, medical score, and/or diagnostic value; and a microprocessor configured to execute at least the decision logic. A viability score may indicate a probability that a specific embryo will result in a live birth, optionally for a specific birth mother. A medical score may represent a probability of a healthy baby, a premature birth, specific medical issues (e.g., preeclampsia), birth defects, and/or the like. The terms “viability score” and “medical score” are not intended to be mutually exclusive.

Various embodiments include a biomedical graph generation system comprising: an input module configured to receive diagnostic data including image data and clinical data; non-transient storage configured to store: a biomedical graph configured for reaching a medical decision based on the diagnostic data, edges of the biomedical graph being configured to represent a decision path through the biomedical graph, nodes of the biomedical graph being configured to receive a segregated subset of the diagnostic data and to provide outputs from a respective node based on analysis of the received segregated subset, one or more neural networks associated with nodes of the biomedical graph, and the diagnostic data; decision logic configured to navigate the biomedical graph based on the diagnostic data and the neural networks, navigation of the biomedical graph resulting in a medical score or diagnostic value; training logic configured to train a neural network, the neural network being associated with a specific node of the biomedical graph, and a microprocessor configured to execute at least the decision logic.

### BRIEF DESCRIPTION OF THE DRAWING

FIGS. 1A-1C illustrates components of a decision system, according to various embodiments of the invention.

FIG. 2 illustrates a dynamic biomedical graph, according to various embodiments of the invention.

FIG. 3 illustrates methods of obtaining an embryo for IVF, according to various embodiments of the invention.

### DETAILED DESCRIPTION

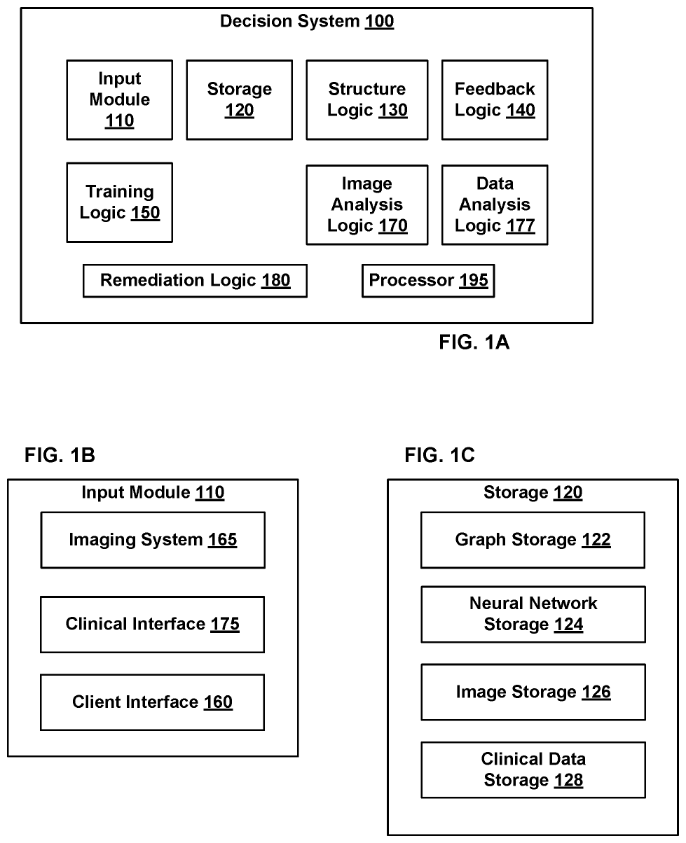

FIGS. 1A-1C illustrate components of a Decision System 100, according to various embodiments of the invention. Decision System 100 may be used in a wide variety of decision-making applications, including medical applications. For the purposes of example, application of Decision System 100 to the improvement and success of IVF (In Vitro Fertilization) is discussed herein. Although, the system is readably applicable to other medical diagnostic needs, it may be adapted to other uses such as: financial analysis, investment analysis, transportation, logistics, diet management, personalized education, network security, structural analysis, and/or the like. Decision System 100 can comprise a single computing system or May be distributed among a plurality of computing system. The “logic” discussed herein includes hardware, firmware, and/or software stored on a non-volatile storage system. For example, the logic can include circuitry and/or integrated circuits configured to perform specific tasks based on stored software instructions.

As shown in FIG. 1A, Decision System 100 includes an Input Module 110. Input Module 110 is configured to received diagnostic data. Diagnostic data can include, data used to determine an issue associated with any complicated or multivariate system. For example, in medical applications, diagnostic data can include: image data, clinical data, medical transcriptions/notes, and/or the like. Image data may include medical images, such as ultrasound images, x-ray images, magnetic imaging images, echocardiography images, elastography images (e.g., ARFI, SSI, SWEI, transient elastography, quasistatic or strain imaging), near-infrared images, fluoroscopy images, radiographs, magnetic particle images, two or three-dimensional images, scintigraphy images, SPECT images, Gamma images, positron emission tomography (PET) images, photographs, video, and/or the like. Image data may include sequences of images taken at various time intervals. For example, video at 2, 8, 12 or 24 frames/second; images taken at intervals of 1, 5, 10, 60, 300, 600 or more seconds; or any range between these values. Image data optionally includes depth and/or three-dimensional information. For example, images may be recorded using a stereoscopic imager, or a camera having selectable focal distances. In one example, image data includes a sequence of images of an embryo recorded over a period of time including one, two, three, four, five or more cell divisions. Such embryo images are optionally recorded at multiple focal depths such that they include three-dimensional information.

In various embodiments, clinical data includes a medical history, data generated using diagnostic tests, drugs and/or treatments provided, genetic modifications made to the embryo or a germ cell of the embryo, chemical exposures (e.g., lead paint, toxins, venom, etc.), trauma history, chemical measurements, clinical events (e.g., a birth or surgery), data from medical devices, socioeconomic data, and or the like. Clinical data may apply to a patient, an embryo, and/or a related party (e.g., a genetic parent of an embryo or a birth mother). Clinical data optionally includes genetic data of one, two or more genetic parents of an embryo, genetic data of a woman who will receive the embryo (birth mother), ages of the genetic parents and/or birth mother, alterations to parent's genetics, height and weight of the genetic parents and/or birth mother, blood tests of the parents/mother, race of the parents/mother, blood tests results, bacterial biome, specific medical conditions (e.g., diabetes, pregnancy history), history of twins, splitting of an embryo, residence location history, and/or any other medically relevant information about an embryo's genetic parents and/or birth mother.

In various embodiments, clinical data includes a chemical history of an embryo, a pH of the embryo, a temperature history of the embryo, an age of the embryo, a chemical analysis of the embryo or embryo environment, spectroscopic analysis of the embryo or embryo environment (absorption, fluorescence, etc.), hormones/drugs/O₂/CO₂/light provided to the embryo, gender of the embryo, genetic modifications made to the embryo, osmotic pressure of embryo cells, number and division of embryotic cells, an internal cell pressure of the embryo, metabolic analysis of embryo culture media, genetic analysis of the culture media, composition of the culture media, presence of volatile organic compounds, and/or the like.

In various embodiments, diagnostic data include caregiver (e.g., doctor) notes and inputs, billing codes, transcriptions of a caregiver statement, and/or the like. In various embodiments, diagnostic data includes digital outputs of medical test equipment, e.g., output of a glucose meter or blood pressure measurement device.

In various embodiments, Input Module 110 includes one or more mechanical device configured to capture the diagnostic data discussed herein, or any combination thereof. For example, Input Module 110 can include an imaging device configured to capture image data associated with an embryo. Specifically, Input Module 110 can include a camera configured to image an embryo during storage and/or initial cell division. Such camera can include those offered in incubators by Esco Medial, Vitrolife or Merck, and can provided a sequence of images of an embryo at any of the frame rates discussed herein.

Input Module 110 optionally includes a user interface configured to receive a caregiver's notes. For example, Input Module 110 can include a graphical user interface for data entry or an API configured to access an electronic medical device and/or to access medical information from an electronic health records system. Input Module 110 optionally includes an instrument interface configured to receive clinical data from a medical device. For example, Input Module 110 may be configured to receive temperature data, spectroscopic data, chemical data, logging data, or other data from a device used to store and/or manipulate embryos.

As shown in FIGS. 1A and 1C, Decision System 100 further includes Storage 120. Storage 120 may include a single device or a plurality of devices. For example, Storage 120 may include non-transient electronic and/or optical storage devices distributed over several locations. Storage 120 may include hard drives, optical drives, random access memory, flash memory, memory circuits, and/or any of the storage media described herein.

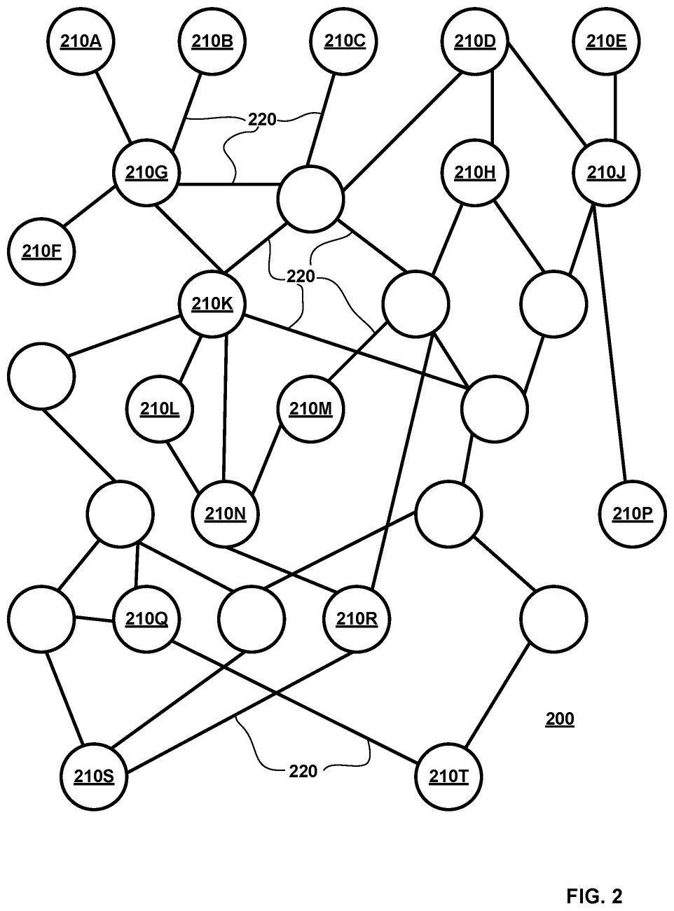

Storage 120 is configured to store least one or more of: Graph Storage 122, Neural Network Storage 124, Image Storage 126 and Clinical Data Storage 128. Graph Storage 122 is configured to store a biomedical graph (such as that illustrated in FIG. 2). More specifically, Graph Storage 122 may be configured to store the data (node identities, edges, state information, metadata, etc.) defining and/or characterizing a biomedical graph. Neural Network Storage 124 is configured to store neural networks. In some embodiments the stored neural networks are each associated with a particular node of the biomedical graph. In other embodiments, at least one of the neural networks is associated with 2, 3, 4 or more nodes of the biomedical graph. Image Storage 126 is configured to store images such as images of embryos. These images may be part of ordered images sequences, e.g., video. Clinical Data Storage 128 is configured to store any of the clinical data discussed herein.

FIG. 2 illustrates a dynamic Biomedical Graph 200, according to various embodiments of the invention. Biomedical Graph 200 includes Nodes 210 (individually designated 210A, 210B, 210C, etc.) and Edges 220 connecting the nodes. Biomedical Graph 200 is an example of a knowledge graph related to biomedical information and/or relationships. The number of Nodes 210 shown are for illustrative purposes. In practice a biomedical graph may include many more nodes. Some of Nodes 210 are “entry nodes” configured as an initial data input point to Biomedical Graph 200, e.g., Nodes 210A-210E. Some of Nodes 210 are “leaf notes” (e.g., Node 210S) configured to be an end point to navigation through Biomedical Graph 200. Some of Nodes 210 are “intermediate nodes” (e.g., Node 210K) through which navigation from entry nodes to leaf nodes occurs. Biomedical Graph 200 is navigated along Edges 220 in a path from entry nodes to leaf nodes. At each node received data is processed to determine a new exit Edge 220. This processing is optionally performed using a neural network or other decision engine specifically associated with the current node. One of Edges 220 may be an “exit edge” from one of Nodes 210 and an “entry edge” from another of Nodes 210, the “exit” and “entry” terminology indicating the direction of graph navigation and/or data flow. In some embodiments, a Node 210 can operate as both a Leaf node and an intermediate node. For example, a node output can be part of the output of Biomedical Graph 200 and also further processed further by other Nodes 210 of Biomedical Graph 200. In a specific example, a Node 210 may output a count of cells in an image of an embryo, that count may be part of the output of Biomedical Graph 200 and may also be used as an input for processing by additional nodes 210 of Biomedical Graph 200.

In an illustrative example, at entry Node 210A input data (e.g., diagnostic data) is received from Clinical Data Storage 128. Optionally, data is received by several entry Nodes 210 in an initial processing step. The division of diagnostic data between more than one entry Nodes 210 represents a segregation of the diagnostic data. As discussed elsewhere herein, Decemberision System 100 optional includes systems and methods of optimizing this segregation between nodes, the identities of nodes, and/or the connections between Nodes 210. At each of the entry Nodes 210 the diagnostic data is processed. This processing can result in selection of an exit Edge 220 from each respective member of entry Nodes 210, and/or of a calculation result that is then passed along the selected exit Edge. The processing at a particular node is optionally performed by a neural network specifically optimized for and associated with the particular node. Thus, one or more of the nodes may each be associated with their own neural network trained for processing data received at entry edges of the node and producing data to be sent at exit edges of the node. Alternatively, one or more of the Nodes 210 may be configured to perform calculations using a script or computer code, rather than a neural network.

Leaf Nodes 210 result in an output indicative of a conclusion that is based on the received diagnostic data and processing by the Biomedical Graph 200. A conclusion may be generated at one or more of leaf Nodes 210. The conclusion may include, for example, a probability of success, a ranking of alternative embryos, an embryo viability or medical score, a suggested treatment (which may be directed at improving an embryo), and/or the like.

The Nodes 210 and Edges 220 of Biomedical Graph 200 represent knowledge about a domain. The connections (Edges 220) between Nodes 210 represent a decision process, which as described elsewhere herein can be dynamically adapted to make better decisions. In some embodiments, Biomedical Graph 200 is configured for reaching a medical decision based on the diagnostic data. In these embodiments, nodes of the biomedical graph are configured to receive a segregated subset of the diagnostic data. For example, Entry Nodes 210A-210E May each be configured to receive and process a particular element of diagnostic data. A result of this processing optionally includes selection of one of Edges 220 exiting each respective entry or intermediate node. The processing may also result in an output that is communicated along the selected edge to the next member of Nodes 210 for further processing and/or output. As such, path or paths taken via Edges 200 through Biomedical Graph 200 represent one or more decision paths through Biomedical Graph 200. In some embodiments, initial nodes are configured to receive one or more images and process these images to generate data characterizing contents of the images. For example, images may be processed to determine cell size, cell growth, cell number, and/or the like.

In some embodiments, images are processed so as to detect changes or kinetics of an embryo. In these cases, changes in the embryo overtime are represented by the output of the Nodes 210 doing the processing. As used herein the term “kinetics” of an embryo is meant to indicate changes that occur overtime. Example outputs include cell division rates, cell growth rates, cell age at division, time between divisions, and/or the like.

Referring to FIG. 1 again, Decision System 100 optionally further comprises structure logic 130. Structure Logic 130 is configured to update a structure of Biomedical Graph 200. This structure can include the introduction or removal of new Nodes 210, and/or the introduction, movement or removal of Edges 220. As such, Structure Logic 130 may be used to reorganize biomedical Graph 200. Such reorganization is typically performed in order to improve the accuracy and/or precision of predictions made using the Biomedical Graph 200. Structure Logic 130 may be responsive to a success of prediction results, viability/medical scores, human input, the availability of new data types, and/or the like. For example, Structure Logic 130 may be used to generate and test random changes in the structure of Biomedical Graph 200, may use various optimization routines to improve Biomedical Graph 200, May employ Monte Carlo techniques to test various biomedical graph structures, and/or the like. For each structure, machine learning systems (e.g., the neural networks associated with each node) may be retrained and the accuracy of predictions tested using test data. Structure Logic 130 is optionally configured to optimize the accuracy of predictions. Optionally, only those machine learning systems proximate two changed Nodes 210 or Edges 220 need to be retrained. For example, in various embodiments, only machine learning systems associated with nodes 1, 2, 3, 5, 7 or fewer edges from a change may be retrained.

Structure Logic 130 is optionally configured to segregate diagnostic data between input Nodes 210 of Biomedical Graph 200. For example, when a new type of data becomes available, Structure Logic 130 may be used to create a new input node to receive this data. Structure Logic 130 is optionally configured to test various segregations of diagnostic data between input Nodes 210 of Biomedical Graph 200. This testing is typically directed at determining more optimal segregations, e.g., segregations that produce better medical predictions. Testing of different data segregations is optionally performed in conjunction with testing of different biomedical graph structures.

Structure Logic 130 is optionally configured to test and discover new correlations between diagnostic data. For example, Structure Logic 130 may discover, using the tests described above, that a combination of specific kinetic data derived from an image sequence and a mother's level of a specific hormone (in combination) makes a particularly good predictor of IVF success. Discovered correlations may be between any diagnostic data, e.g., combinations of image generated data and/or clinical data. The identification of data that provides good predictive results can sometimes lead to the identification of clinical steps (e.g., raising a hormone level or adjusting a salinity of an embryo's environment) that can result in improved medical outcomes.

In some embodiments, Structure Logic 130 is configured to receive feedback or other input from a human user and to modify the structure of Biomedical Graph 200 and/or data segregation based on this input. For example, a user may designate that each of a sequence of images should be provided to an entry Node 210 and the outputs of these Nodes 210 (possibly including cell count and/or size data) be provided to an intermediate Node 210 configured to derive kinetic information from the outputs of the entry Nodes 210.

Decemberision System 100 optionally further comprises Feedback Logic 140. Feedback Logic 140 is configured to receive and/or generate a score characterizing an output generated at one or more Nodes 210 of Biomedical Graph 200. For example, a score may be representative of an output of a particular Node 210 and a decision to select one of several alternative output Edges 220 from that Node 210. In another example, a score is representative of the performance of a group of Nodes 210 or the entire Biomedical Graph 200. Scores may be based on an input from a human user (e.g., a manually entered value), or be based on a clinical result. Specifically, a score may be based on an accuracy of a prediction that an embryo will result in a successful pregnancy, an accuracy of a ranking between alternative embryos, and/or a statistical analysis of the accuracy of an output of Biomedical Graph 200.

Scores generated using Feedback Logic 140 are optionally used by Structure Logic 130 to identify Nodes 210 and/or Edges 220 that may be modified to improve the performance of Biomedical Graph 200. In a specific example, a group of Nodes 210 connected by Edges 220 may be configured to each process images of an embryo at different times and determine kinetic data (e.g., time between cell divisions) from these images. The output of these nodes may be compared with data collected manually and a score calculated therefrom.

Decision System 100 optionally further comprises Training Logic 150. Training Logic 150 is configured to train machine learning systems (e.g., neural networks) associated with Biomedical Graph 200. For example, Training Logic 150 may be configured to train an individual neural network associated with a particular member of Nodes 210, or group of Nodes 210. Training Logic 150 may use any of the known methods of neural network training. Training Logic 150 is optionally configured to train a set (1 or more) of neural networks associated with a subset of Nodes 210 of Biomedical Graph 200. Optionally, Training Logic 150 is configured to train individual neural networks, without requiring retraining of all the neural networks associated with Nodes 210 of Biomedical Graph 200. Training Logic 150 may be configured to train sets of neural networks for specific tasks such as analysis of images to measure morphological and/or kinetics of embryos.

Decemberision System 100 optionally further comprises a Client Interface 160. In various embodiments Client Interface 160 includes logic configured to present results on a display device such as a mobile device or computer screen. Client Interface 160 may be presented via an application or a webpage. Client Interface 160 is configured to report a result of navigating the biomedical graph to a user. In one mode, the report may include just a result such as a probability of success for a specific embryo, optionally represented by the outputs of one or more leaf Nodes 210. Alternatively, the report may include a description of the path taken through Biomedical Graph 200 and/or outputs of entry or intermediate Nodes 210. A report including the path taken can include a basis or explanation for the output generated. As noted elsewhere herein, the output can include a probability and/or a relative score (between alternative embryos). Optionally, Client Interface 160 is configured to provide suggestions for improving the probability of success for an embryo (i.e., a successful pregnancy). Such suggestions can include, for example, a change in conditions under which the embryo is stored, a hormone treatment for a mother, an embryo age at implantation, and/or the like. Such suggestions may be generated by Remediation Logic 180, discussed elsewhere herein.

In an illustrative embodiment, Client Interface 160 is enabled included in a mobile application which is configured to provide a regular update to a user regarding the status of one or more embryos. The update can include, for example, embryo photos/video, quantitative measures of the embryos, relative embryo ratings, embryo scores, and/or the like. The quantitative measure may be determined using Nodes 210 of Biomedical graph. In this embodiment, the user may be provided with regular (e.g., daily) updates regarding embryo growth, cell division, days to implantation, etc. Different versions of Client Interface 160 May be provided to patients and caregivers.

Decision System 100 optionally further comprises Image Analysis Logic 170. Image Analysis Logic 170 is configured to generate clinical data from one or more images, e.g., an image sequence. Image Analysis Logic 170 may or may not be include neural networks within one or more of Nodes 210. For example, in some embodiments, Image Analysis Logic 170 includes a neural network that is separate from Biomedical Graph 200. In this case, the neural network may be configured to process images and generate an output including characteristics in those images. This output is then optionally provided to input Nodes 210 of Biomedical Graph 200. The processed images are optionally part of a sequence of images recorded overtime. For example, the images may be recorded at time intervals discussed elsewhere here in. Alternatively, Image Analysis Logic 170 may include one or more neural networks included in Nodes 210 of Biomedical Graph 200.

Outputs of Image Analysis Logic 170 can include any combination of the image characteristics discussed herein, including: kinetic characteristics, cell size, time between cell divisions, cell age, cell count, cell spectroscopic characteristics (e.g., fluorescence, absorption & wavelength dependent features), three-dimensional structure of an embryo, cell uniformity (with regard to any of the characteristics), and/or the like. In a specific example, the output of Analysis logic 170 includes a count of specific cells within an embryo that have failed to divide within a specific time period, and/or a count of cells that have divided in that period.

The output of Image Analysis Logic 170 can include any features identified in the image(s), and/or may include kinetic data representative of changes in identified features over time. These features can include, for example, cell size, cell number, cell depth, cell packing, cell division, cell differentiation, cell adhesion, or any other feature determined (optionally by a neural network as discussed herein) as being indicative of viability for an embryo. In various embodiments, Image Analysis Logic 170 is configured to generate clinical data including cell count, cell division intervals, cell movement, cell morphology, cell size, mitosis rate, cell uniformity, cell spectroscopy (wavelength dependent), cell growth state characteristics (M, G1, S, G2, G0), cell division paths (are all cells dividing), individual cell tracking, cell metabolism markers, cell index of refraction, and/or the like.

Decemberision System 100 optionally further comprises Data Analysis Logic 177. Data Analysis Logic 177 is configured to analyze clinical data and generate derivative data that can be processed by Biomedical Graph 200. For example, Data Analysis Logic 177 may be used to calculate an average of a mother's hormone levels, to generate a summary of a mother's diet or physical activity. Data Analysis Logic 177 may be configured to perform the analysis of clinical data using mathematical models (e.g., data annotation, correlation analysis, statistical analysis, smoothing routines, optimization routines, etc.), natural language processing (of medical notes), transform narrative data to a numerical form, and/or the like. In general, Data Analysis Logic 177 may be configured to convert non-image data into a form better structured for processing by Biomedical Graph 200.

As described above with respect to Image Analysis Logic 170, Data Analysis Logic 177 may or may not include neural networks associated with Nodes 210 of Biomedical Graph 200.

Decemberision System 100 optionally further comprises Remediation Logic 180. Remediation Logic 180 is configured to suggest a change in diagnostic data likely to lead to an improved probability of success for an embryo. As noted elsewhere herein, success for an embryo includes a successful pregnancy and birth. Specifically, Remediation Logic 180 may be identified to identify remediations (e.g., changes or treatments) that would improve success. Such remediations may include giving a mother a drug, changing environmental or chemical conditions for an embryo (e.g., a greater temperature, light or nutrient supply, or providing a mitosis promoter), modification of embryo DNA, allowing additional time before embryo implantation, removal of a subset of embryo cells (e.g., ones not dividing), and/or the like.

In some embodiments, Remediation Logic 180 is configured to test the effect of changing diagnostic data provided to Biomedical Graph 200 to determine what changes could improve embryo success. Where practical, steps to accomplish these modifications may be implemented.

Decemberision System 100 optionally further comprises one or more Processor 195. Processor 195 is a digital, electronic, and/or optical microprocessor configured to execute at least logic included in Biomedical Graph 200, Structure Logic 130, and/or Image Analysis Logic 170.

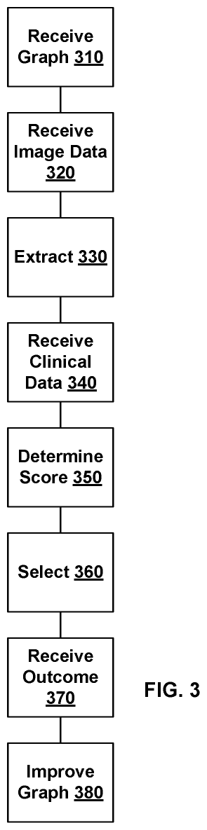

FIG. 3 illustrates methods of obtaining and/or selecting an embryo for IVF, according to various embodiments of the invention. These methods may be used to select from among a plurality of alternative embryos for implantation in the uterus of a patient, to determine a number of embryos to implant in a particular IVF cycle, to decide to create additional embryos, to control a probable multiplicity of a pregnancy (e.g., probably of singlet, twins, triplets, etc.), to determine optimum characteristics of and/or selection of a surrogate, and/or to guide treatments (e.g., hormone treatments) of the egg source, sperm source, and/or surrogate), and/or any combination thereof. The methods illustrated by FIG. 3 are optionally used for applications other than IVF.

In an optional Receive Graph Step 310, a Biomedical Graph 200 is received. Receive Graph Step 310 is optional in embodiments wherein the Biomedical Graph 200 is generated from scratch or managed by a third party. As discussed elsewhere herein, the biomedical graph is configured for reaching a medical decision based on the diagnostic data, edges of the biomedical graph being configured to represent a decision path through the biomedical graph, nodes of the biomedical graph being configured to receive a segregated subset of the diagnostic data and to provide outputs from a respective node based on analysis of the received segregated subset. In specific embodiments, the received Biomedical Graph 200 is configured for generating viability and/or medical scores for embryos for use in IVF. The received Biomedical Graph 200 is optionally stored in Storage 120.

In a Receive Image Data Step 320, image data of an embryo is received. This image data can include an ordered sequence of images recorded over any of the time periods discussed herein, e.g., over a period of at least 1, 2, 3, 5, 7 10, 14 or 21 days, or any range there between. The number of images included in a sequence may vary widely, e.g., from more than a frame per second, a frame per minute, a frame per 10 minutes, a frame per hour, a frame every 3, 6, 12, 18 or 24 hours, or any range therebetween. The image data is optionally obtained using Imaging System 165 while the embryo is in a cold storage container.

In an Extract Step 330, clinically relevant features of the embryo are extracted from the received image data. Extract Step 330 is optionally performed by Image Analysis Logic 170 and/or a trained neural network. The extracted features can include any of the embryo characteristics discussed herein, which can be extracted from image data. The identity of relevant features to extract is optionally determined by Image Analysis Logic 170, Feedback Logic 140, Data Analysis Logic 177, and/or other elements of Decision System 100.

In a Receive Clinical Data Step 340, clinical data associated with the embryo is received. The clinical data may include any of the clinical data discussed herein and may be related to genetic parents of the embryo and/or a prospective birth mother into whom the embryo is to be implanted. Together the features extracted in Extract Step 330 and the clinical data received in Clinical Data Step 340 constitutes diagnostic data.

In some embodiments the clinical data received in Receive Clinical Data Step 340 includes prospective information. For example, the data may include the administration of a hormone or timing of embryo insertion that has not yet occurred. Use of such data may be used to answer “what if” questions and to guide medical decision making. For example, a viability of the embryo may be different if inserted into the uterus on day X as compared to day Y. These two insertion days may be received in Receive Clinical Data Step 340 as alternatives, and any changes in the probability of a live birth resulting from these alternatives may be estimated. By using alternative data in the methods of FIG. 3 medical caregivers (and patients) can make more informed treatment decisions. Consequences of any of the controllable medical characteristics, procedures and/or treatments discussed herein may be thus examined.

In a Determine Score Step 350, a score for the embryo is determined. This score may be a medical score and/or a viability score. As is discussed elsewhere herein, the determination of the score may be dependent on the diagnostic data and navigation of a biomedical graph, such as Biomedical Graph 200. The score may be dependent on characteristics of any of the parties involved (birth mother, genetic parents, etc.), on past medical treatments, on prospective medical treatments, and/or any other information discussed herein. Determine Score Sep 350 is optionally performed using elements of Decision System 100 as discussed herein, for example, using Data Analysis Logic 177 and neural networks stored in Neural Network Storage 124. The determined score is optionally representative of a probability that the embryo will result in a live birth. The determined score is optionally a comparative score configured to compare viability of alternative embryos and/or to compare consequences of different medical treatments (and/or events).

In a Select Step 360, one or more embryo for use in IVF is selected from among a plurality of alternative embryos based on the (medical and/or viability) score. Alternatively, or additionally, one or more medical treatment is selected based on the score. For example, in some embodiments, Select Step 360 includes selecting a quantity of embryos to implant in a uterus in a single IVF cycle based on the viability score of each of the embryos, and optionally based on a target multiplicity of a pregnancy. In another example, a date (e.g., since fertilization) for implantation in a uterus, and/or a number of cell divisions required before implantation, is selected based on relative viability scores. For example, Data Analysis Logic 177 (and/or other decision logic of Decision System 100) may be configured to calculate a probability distribution for a multiplicity of a pregnancy based on viability scores of a plurality of embryos inserted into a uterus in a single IVF cycle. Such a distribution may include a probability of no birth, a probability of a single birth, and probabilities of twins, triplets, quadruplets, etc. Select step 360 optionally includes selection of specific embryos and/or quantities thereof to achieve a desired probability distribution of birth multiplicity. In some embodiments, Select Step 360 includes deciding to create additional embryos based on a viability score of an existing embryo.

In an optional Receive Outcome Step 370, the outcome of an IVF cycle for which the embryo was used is received. This outcome can include data such as: was there a live birth, if so, how many, were there pregnancy complications, was the birth premature, was the baby healthy, etc. In an optional Improve Graph Step 380, the outcome received in Receive Outcome step 370 is used to improve the Biomedical Graph 200 and/or neural networks of Decision System 100. As discussed elsewhere herein, these improvements can include changing the structure of the biomedical graph, the changes to the structure including adding, eliminating or moving nodes, or changing connections between nodes. Further, these improvements can include training neural networks associated with nodes of the biomedical graph, e.g., using Training Logic 150.

Several embodiments are specifically illustrated and/or described herein. However, it will be appreciated that modifications and variations are covered by the above teachings and within the scope of the appended claims without departing from the spirit and intended scope thereof. For example, while the prediction of embryo success in IVF is used herein as an example, the systems and methods discussed herein may be applied to other medical and non-medical applications. These include any of the other applications discussed herein.

The embodiments discussed herein are illustrative of the present invention. As these embodiments of the present invention are described with reference to illustrations, various modifications or adaptations of the methods and or specific structures described may become apparent to those skilled in the art. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

Computing systems and/or logic referred to herein can comprise an integrated circuit, a microprocessor, a personal computer, a server, a distributed computing system, a communication device, a network device, or the like, and various combinations of the same. A computing system or logic may also comprise volatile and/or non-volatile memory such as random-access memory (RAM), dynamic random-access memory (DRAM), static random access memory (SRAM), magnetic media, optical media, nano-media, a hard drive, a compact disk, a digital versatile disc (DVD), optical circuits, and/or other devices configured for storing analog or digital information, such as in a database. A computer-readable medium, as used herein, expressly excludes paper. Computer-implemented steps of the methods noted herein can comprise a set of instructions stored on a computer-readable medium that when executed cause the computing system to perform the steps. A computing system programmed to perform particular functions pursuant to instructions from program software is a special purpose computing system for performing those particular functions. Data that is manipulated by a special purpose computing system while performing those particular functions is at least electronically saved in buffers of the computing system, physically changing the special purpose computing system from one state to the next with each change to the stored data.

The “logic” discussed herein is explicitly defined to include hardware, firmware or software stored on a non-transient computer readable medium, or any combinations thereof. This logic may be implemented in an electronic and/or digital device (e.g., a circuit) to produce a special purpose computing system. Any of the systems discussed herein optionally include a microprocessor, including electronic and/or optical circuits, configured to execute any combination of the logic discussed herein. The methods discussed herein optionally include execution of the logic by said microprocessor(s).

What is claimed is:

1. A medical decision system comprising:
an input module configured to receive diagnostic data including image data and clinical data;
non-transient storage configured to store:
a biomedical graph configured for reaching a medical decision based on the diagnostic data, edges of the biomedical graph being configured to represent a decision path through the biomedical graph, nodes of the biomedical graph being configured to receive a segregated subset of the diagnostic data and to provide outputs from a respective node based on analysis of the received segregated subset,
one or more neural networks associated with nodes of the biomedical graph, and
the diagnostic data;
decision logic configured to navigate the biomedical graph based on the diagnostic data and the neural networks, navigation of the biomedical graph resulting in a viability score, medical score or diagnostic value; and
a microprocessor configured to execute at least the decision logic.

2. A biomedical graph generation system comprising:
an input module configured to receive diagnostic data including image data and clinical data;
non-transient storage configured to store:
a biomedical graph configured for reaching a medical decision based on the diagnostic data, edges of the biomedical graph being configured to represent a decision path through the biomedical graph, nodes of the biomedical graph being configured to receive a segregated subset of the diagnostic data and to provide outputs from a respective node based on analysis of the received segregated subset,
one or more neural networks associated with nodes of the biomedical graph, and
the diagnostic data;
decision logic configured to navigate the biomedical graph based on the diagnostic data and the neural networks, navigation of the biomedical graph resulting in a viability score, medical score, and/or diagnostic value;
training logic configured to train a neural network, the neural network being associated with a specific node of the biomedical graph.
a microprocessor configured to execute at least the decision logic.

3. A method of obtaining or selecting an embryo for IVF, the method comprising:
receiving image data of an embryo;
extracting features of the embryo from the received image data;
receiving clinical data related to the embryo, the extracted features and the clinical data being diagnostic data;
determining a viability score for the embryo, the determination of the viability score being dependent on the diagnostic data and navigation of a biomedical graph; and
selecting the embryo for use in IVF based on the viability score.

4. The method of claim 3, further comprising receiving the biomedical graph, the biomedical graph being configured for reaching a medical decision based on the diagnostic data, edges of the biomedical graph being configured to represent a decision path through the biomedical graph, nodes of the biomedical graph being configured to receive a segregated subset of the diagnostic data and to provide outputs from a respective node based on analysis of the received segregated subset.

5. The method of claim 3, further comprising receiving an outcome for the embryo, and improving the biomedical graph based on the received outcome.

6. The method of claim 5, wherein improving the biomedical graph includes changing the structure of the biomedical graph, the changes to the structure including adding, eliminating or moving nodes, or changing connections between nodes.

7. The method of claim 5, wherein improving the biomedical graph includes training neural networks associated with nodes of the biomedical graph.

8. The method of claim 3, wherein the viability score is representative of a probability that the embryo will result in a live birth.

9. The method claim 3, further comprising selecting a quantity of embryos to implant in a uterus in a single IVF cycle based on the viability score and a target multiplicity of a pregnancy.

10. The system of claim 1, wherein the decision logic is configured to calculate a probability distribution for a multiplicity of a pregnancy based on viability scores of a plurality of embryos inserted into a uterus in a single IVF cycle.

11. The system of claim 1, wherein the image data includes a sequence of images of an embryo recorded over a period of time including at least two cell divisions.

12. The system of claim 1, wherein the clinical data includes genetic data of one or two genetic parents of an embryo, or genetic data of a woman who will receive the embryo (birth mother).

13. The system claim 1, wherein the clinical data includes a chemical history of an embryo, a pH of the embryo, a temperature history of the embryo, an age of the embryo, a chemical analysis of the embryo or embryo environment, hormones/drugs/O₂/CO₂/light provided to the embryo, sex of the embryo, or genetic modifications made to the embryo.

14. The system of claim 1, wherein intermediate nodes of the biomedical graph each include one or more input edge and one or more output edge, the nodes being each associated with a respective member of the one or more neural networks configured to take data from the one or more input edges as input and to provide the outputs to the one or more output edges, each of the input edges and output edges of an intermediate node connecting to a different node of the biomedical graph.

15. The system of claim 1, wherein the decision logic is configured to provide a viability score for an embryo.

16. The system of claim 1, wherein the decision logic is configured to rank viability of two or more embryos.

17. The system of claim 1, wherein the decision logic is configured to provide an output based on diagnostic data associated with a specific individual or pair of individuals.

18. The system of claim 1, further comprising structure logic configured to update a structure of the biomedical graph, the structure including nodes of the biomedical graph and edges between the nodes.

19. The system of claim 18, wherein the structure logic is configured to segregate the diagnostic data between input nodes of the biomedical graph.

20. The system of claim 18, wherein the structure logic is configured to test various biomedical graph structures.

21. The system of claim 18, wherein the structure logic is configured to test various segregations of the diagnostic data between input nodes of the biomedical graph and to determine more optimal segregations based on the test of various segregations.

22. The system of claim 18, wherein the structure logic is configured to discover new correlations between diagnostic data.

23. The system of claim 1, further comprising image analysis logic configured to generate at least part of the clinical data from one or more images.

24. The system of claim 23, wherein the image analysis logic is configured to generate clinical data including cell count, cell division intervals, cell movement, cell morphology, cell size, mitosis rate, cell uniformity, cell spectroscopy, cell growth state characteristics, cell division paths, individual cell tracking, cell metabolism markers, or cell index of refraction.

25. The system of claim 1, wherein the image data includes one or more photographs of an embryo or a video of the embryo.

26. The method of claim 3, wherein the image data includes one or more photographs or a video of the embryo.

27. The method of claim 3, wherein the clinical data includes a chemical history of the embryo, a pH of the embryo, a temperature history of the embryo, an age of the embryo, a chemical analysis of the embryo or embryo environment, hormones or drugs or O₂ or CO₂ or light provided to the embryo, sex of the embryo, or genetic modifications made to the embryo.

28. The method of claim 3, wherein the viability score is configured to rank viability of two or more embryos.
